(19) United States
(12) Patent Application Publication
(10) Pub. No.: US 2016/0146833 A1
(43) Pub. Date: May 26, 2016

(54) STRESS AND INFLAMMATION BIOMARKER URINE PANEL FOR DAIRY COWS AND BEEF CATTLE

(71) Applicant: WELLMETRIS LLC, Keego Harbor, MI (US)

(72) Inventors: Andrew A. Dahl, Bloomfield Hills, MI (US); Patrick Kincaid, Macomb, MI (US); Myron C. Rapkin, Indianapolis, IN (US)

(21) Appl. No.: 14/904,274

(22) PCT Filed: Jun. 16, 2014
(86) PCT No.: PCT/US14/42464

§ 371 Date: Jan. 11, 2016

Related U.S. Application Data
(60) Provisional application No. 61/835,282, filed on Jun. 14, 2013.

(51) Int. Cl. G01N 33/68; G01N 33/84; G01N 33/88

(52) U.S. Cl. CPC G01N 33/6893; G01N 33/88; G01N 33/6869; G01N 33/6863; G01N 33/84; G01N 2800/7095; G01N 2800/7009; G01N 2800/70; G01N 2800/60; G01N 2333/51; G01N 2333/525; G01N 2333/5412; G01N 2333/5421; G01N 2333/765; G01N 2333/4713

(57) ABSTRACT

A panel for monitoring levels of biomarkers in ruminants, including an assay having at least one inflammation monitoring test, at least one oxidative stress monitoring test, and at least one antioxidant activity monitoring test. A method of monitoring the health of ruminants, by collecting a sample from the ruminant, applying the sample to an assay panel, performing at least one inflammation monitoring test, at least one oxidative stress monitoring test, and at least one antioxidant activity monitoring test in the panel, and determining levels of biomarkers related to inflammation, oxidative stress, and antioxidant activity and therefore providing information regarding the ruminant's relative health and/or risk of developing one or more diseases.

### BACKGROUND OF THE INVENTION

1. Technical Field

The present invention relates to assays and panels for detection of biomarkers in ruminants, with the term “biomarker” referring to an analyte in a sample that is associated with a physiological condition and/or the presence or risk of contracting one or more diseases. In particular, the present invention relates to non-invasive detection of biomarkers in urine of ruminants.

2. Background Art

Cattle are not sedate, cud-chewing biomass processors. They are, in fact, very easily stressed and subject to runaway inflammation cascades—a genetic relic of their life prior to domestication. As such, producers are constantly monitoring the health of cattle, trying to stay ahead of infective agents, overcrowding, and changes in routine that can have a profound effect on milk production or beef yield.

Bovine mastitis, the inflammation of the udder due to infection or physical insult, is the number one cause of lost milk production in the US and worldwide. In the US, lost milk production exceeds 845 lbs. per cow, per year. There are 9 million dairy cows in the US. Therefore, the amount of lost milk is roughly 7.6 billion pounds per year—a $3 billion problem for the dairy industry.

Detecting pre-conditions to mastitis would allow the dairy producer to intervene before a mastitis flare-up, which requires antibiotic treatment and subsequent withdrawal of milk product for at least several days.

Transition cow syndrome is another costly production issue affecting first-time milkers. The stress of the first lactation can cause the dairy cow's immune system to shut down, opening the door to disease. Not all cows succumb to transition cow syndrome, yet there is no simple urine-based field test to determine if the cow is under metabolic stress so that action can be taken. By the time clinical signs emerge, the cow is well into the syndrome and mastitis has likely flared.

Bovine respiratory disease complex (BRDC) aka “shipping fever” affects beef cattle that have been rounded up at a free-range ranch and trucked to a feedlot. The stress of the roundup, trucking and crowded condition of the feedlot cause some cattle to lose as much as 30% of their body weight. In the US, beef producers can lose up to $10 billion per year due to BRDC. However, as with dairy cows, it's very difficult to detect the complex early enough to intervene in a cost and time-effective manner.

It is well established in the scientific literature that certain physiological conditions, including oxidative stress and/or chronic inflammation, play key roles in several pathological disturbances such as atherosclerosis, obesity, diabetes, neurodegenerative diseases and cancer. Diet, lifestyle, exercise, as well as certain drugs have anti-inflammatory and/or anti-oxidant activity. Indeed, the market for antioxidants alone runs to billions of dollars per year. Many biomarkers for inflammation, oxidative stress, and anti-oxidant activity have been reported in the literature.

In contrast to the assessment of wellness or relative health, or for the assessment of the risk of development of disease(s), traditional tests are designed and employed to diagnose specific diseases, with an increasing emphasis on early diagnosis. Some available tests do analyze for some substances, such as cholesterol, lipoproteins, and CRP (c-reactive protein), albumin/creatinine ratio, and some other “risk factors” for specific diseases, e.g. cardiovascular disease. But, the disease-specific application of these few pre-symptomatic tests is still consistent with traditional medicine's focus on biomarkers for the diagnosis of specific disease

For example, although chronic inflammation is associated with a significant increase in the risk for certain cancers, and regular use of drugs or dietary agents with anti-inflammatory activity have been proven to reduce the risk for such cancers, traditional clinical laboratories and clinicians do not monitor biomarkers for inflammation as risk factors for cancer.

Some “esoteric laboratories” offer a large number of tests such as cytokine assays, mostly using blood samples, to test for many reported biomarkers associated with disease(s) or disease risk. A few internet-based companies offer products that are purported to provide for the qualitative determination of oxidative stress biomarkers such as TBARS (thiobarbituric acid reactive substances) or other tests for biomarkers associated with oxidative stress (e.g. isoprostanes) in urine.

However, with the exception of the disease-specific (almost exclusively related to cardiovascular disease) application of the few examples cited above, at present none are readily available to individuals seeking to determine how healthy (low inflammation, low oxidative stress, high antioxidant activity) they are. As a specific example, the currently available CRP test only interprets the level of CRP as a marker for cardiovascular risk.

A few companies offer a wide range of exotic tests for human physiological biomarkers. For example, Genova Diagnostics offers an inflammation panel comprised of 3 inflammatory biomarkers (hsCRP, homocysteine and fibrinogen) in a blood sample, and an Oxidative Stress 2.0 blood test panel comprised of 10 biomarkers, one of which is lipid hydroperoxides. However, typically these tests are run either individually or in panels on blood samples and almost always require the samples be sent to a core laboratory. The latter requirement introduces several undesirable characteristics, including: the time, effort and cost of collection and transport of the specimens, the significant potential for ex vivo changes in the level(s) of the analytes that may arise either from the decomposition of an analyte or the artifactual generation of additional analyte from precursors in a sample. Such artifactual ex vivo changes in the levels of analytes are particularly well known in the case of oxidative stress biomarkers, but can also occur for inflammatory biomarkers in blood or urine specimens. For example, isoprostanes, which are well-studied biomarkers of oxidative stress, are rapidly generated ex vivo by the action of reactive oxygen species on arachidonic acid present in blood samples; and the level of protein in a urine sample may artifactually increase within hours at room temperature due to bacterial growth.

For example, U.S. Pat. No. 6,953,666 to Kinkade, Jr., et al. discloses methods and compositions for detecting the presence of oxidized derivatives of amino acids in proteins as biomarkers of oxidative stress. In principle, the biomarker can be any amino acid that has undergone oxidation (or other modification, e.g. dityrosine, nitrotyrosine which is produced by the reaction of tyrosine with peroxynitrite, or chloro-tyrosine, which is produced by the action of myeloperoxidase and is an inflammatory biomarker). Emphasis in Kinkade, Jr., et al. is given to oxidized sulfur- or selenium-containing amino acids (SSAA). Oxidized SSAA are amino acids in which the sulfur or selenium moiety has been oxidized to some oxidation state. Oxidized SSAA include, but are not limited to, cysteine, cystine, methionine, selenomethionine, selenocystine and selenocysteine in their various possible oxidation states. Typically, an ELISA assay is provided for quantification of these biomarkers.

U.S. Pat. No. 6,852,541 to Obayan, et al. discloses an assay for testing oxidative stress of a subject by measurement of oxidants in biological fluids such as urine, plasma, bioreactor medium and respiratory aspirants. There is provided a method of determining oxidative stress in a mammalian subject. The method comprises: obtaining a sample of a biological fluid from the subject; mixing the biological fluid with a ferrous reaction reagent; incubating the biological fluid and the reaction reagent; and detecting a colored reaction product. There is further provided a ferrous reaction reagent suitable for use in assaying oxidative stress, said reaction reagent comprising 2-deoxyglucose, TBA, EDTA, and ferrous sulfate, and being substantially free of ascorbic acid.

U.S. Pat. No. 7,288,374 to Pincemail, et al. discloses a process for detecting oxidative stress in a sample and to a kit for this implementation. According to one embodiment, the Pincemail, et al. invention provides a method for the detection of oxidative stress in an individual carrying a risk factor for oxidative stress comprising determining the risk factor for oxidative stress of said individual; selecting at least two oxidative stress markers being increased or decreased for said risk factor relative to healthy individuals; and measuring the amount of said at least two oxidative stress markers in a sample obtained from said individual. Oxidative stress markers in the invention of Pincemail, et al. are detected from whole blood samples or samples containing components thereof.

U.S. Pat. No. 5,858,696 to Roberts, II et al. discloses a method of assessing oxidative stress in vivo by quantification of prostaglandin F2-like compounds and their metabolites produced by a non-cyclooxygenase free radical catalyzed mechanism.

U.S. Pat. No. 5,912,179 to Alvarez, et al. discloses systems and methods for material analysis in which an organic sample (e.g., a foodstuff, tissue sample or petroleum product) is illuminated at a plurality of discrete wavelengths that are absorbed by fatty acid and fatty acid oxidation products in the sample. Measurements of the intensity of reflected or absorbed light at such wavelengths are taken, and an analysis of absorbance ratios for various wavelengths is performed. Changes in the reflection ratios are correlated with the oxidative state of fatty acids present in the material.

U.S. Pat. Nos. 6,096,556 and 6,133,039 disclose a non-invasive method for the determination of oxidative stress in a patient by urinalysis. The method comprises quantifying the level of o,o′-dityrosine in a sample of the urine of the patient and comparing with the corresponding level of the compound in a normal or control sample, whereby a substantially elevated level of said o,o′-dityrosine is indicative of oxidative stress in the patient.

U.S. Pat. No. 6,541,265 to Leeuwenburgh discloses methods and systems for testing a substance for inflammatory or oxidant properties under acute inflammatory conditions characterized by increased levels of redox-active metal ions. The method includes the steps of applying an eccentric exercise stimulus to a subject, thereby inducing a muscle injury; administering a substance of interest to the subject; measuring one or more biological markers of inflammation, oxidative stress, and muscle damage, or combinations thereof, within the subject; and correlating the measured value of the biological marker(s) with the inflammatory or oxidative properties of the substance administered. The systems of the subject invention include means for obtaining a biological sample from a subject, means for applying eccentric exercise stimulus to the subject; means for measuring the amount of the biological marker(s) within the biological sample; and means for correlating the measured amounts of the biological marker(s) with the inflammatory or oxidant properties of the substance administered.

U.S. Pat. No. 6,569,683 to Ochi, et al. discloses a diagnostic plot derived from the measurement of 82 assays that characterize two key parameters that significantly contribute to an individual's health status. These two parameters are oxidative stress profile (OSP) and antioxidant profile. Each of the 82 assays is complimentary with other assays of the profile, thus providing either confirmation information or the synthesis of new information. The diagnostic plot, developed to interpret the assay data, which provides information about oxidative damage and antioxidant protection, consists of four quadrants, each with noticeable characteristics. By visually assessing the position of a patient's OSP status, in comparison to reference OSP values in the four quadrants constituting the diagnostic plot, physicians and other health care professionals can provide sound advice to their patients regarding dietary and life style changes one need to adhere for prevention of oxidative stress-related diseases as well as postponing premature aging processes.

Vassalle et al. (Vassalle C, Pratali L, Boni C, Mercuri A, Ndreu R. An oxidative stress score as a combined measure of the pro-oxidant and anti-oxidant counterparts in patients with coronary artery disease. Clin Biochem. 41:1162-7 (2008)) have report an “oxidative stress index” in which tests for both the oxidative damage and antioxidant components of a blood sample are performed and the Oxidative-INDEX is computed based on a formula employing both components.

U.S. Patent Application Publication No. 2007/0054347 to Rosendahl, et al. discloses an optical analyzer for measuring an oxidative stress component in a patient, having a light source and a light detector used for measuring an optical property of a medium and generating optical measurement data. A processor analyzes the optical measurement data and generates a value for one or more oxidative stress component in the form of a redox signature for the patient. Probability data of the presence of an oxidative stress dependent disease can be calculated. By observing at least one additional clinical condition of the disease, a diagnosis using said at least one additional condition and said redox signature can be obtained.

U.S. Patent Application Publication No. 2010/0267037 to Westbrook, et al. discloses a method for detection of inflammatory disease in a subject that comprises assaying a test sample of peripheral blood from the subject for a marker of DNA damage. An elevated amount of the marker present in the test sample compared to control sample and this is described to be indicative of inflammatory disease activity, including sub-clinical inflammation. The method can be adapted for quantitatively monitoring the efficacy of treatment of inflammatory disease in a subject. Markers of DNA damage include single- and/or double-stranded breaks in leukocytes, oxidative DNA damage in leukocytes, or a marker of nitric oxide oxidative activity (protein nitrosylation in leukocytes). The inflammatory disease can be inflammatory bowel disease (ulcerative colitis or Crohn's disease). The invention is described as also being useful for detection of other types of inflammatory disease, such as non-immune intestinal inflammatory disease (diverticulitis, pseudomembranous colitis), autoimmune diseases (rheumatoid arthritis, lupus, multiple sclerosis, psoriasis, uveitis, vasculitis), or non-immune lung diseases (asthma, chronic obstructive lung disease, and interstitial pneumonitis).

The methods cited above typically require complex instrumentation and technically skilled operator, so that they are expensive and not suitable for widespread application. Further, as noted above, this typically requires that samples be transported to specialized locations capable of performing such analyses, which may result in alterations to the analyte(s).

Many devices have been developed to analyze for specific substances in biological specimens at the point of testing by employing dry chemical, microfluidic and/or immunochemical methods. Several such methods, which are in widespread use, are essentially dry chemistry tests involving test pads into which chemicals have been impregnated and which react relatively specifically with analytes in with biofluids, and the results of which can be read by optical or other methods. The analysis can involve simply visual comparison to the color of a reference chart, which is widely employed for the qualitative analysis of water in pools and spas and for the analysis of multiple disease-related analytes in urine and other body fluids. Semi-quantitative results may be obtained by the application of a device to measure the amount of color developed.

For example, U.S. Pat. No. 5,597,532 to Connolly discloses an apparatus for the optoelectronic evaluation of test paper strips for use in the detection of certain analytes in blood or other body fluids. The test strip comprises an elongated plastic part including a hinged portion to allow a first portion to be folded over a second portion. A series of layers of test strips are disposed between the folded over portions of the test strip. The test strip is configured such that the chemistry layers are placed in contacting engagement with one another, but not compressing one another. A reflectance photometer is provided and includes various features, including a lot number reader wherein if the test strip does not match the memory module, a test is not performed, and the user is instructed to insert a correct memory module.

U.S. Pat. Nos. 6,511,814 and 6,551,842 to Carpenter discloses a disposable, dry chemistry analytical system that is broadly useful for the detection of a variety of analytes present in biological fluids such as whole blood, serum, plasma, urine and cerebral spinal fluid. The invention discloses the use of the reaction interface that forms between two liquids converging from opposite directions within a bibulous material. The discovery comprises a significant improvement over prior art disposable, analytical reagent systems in that the detectable reactant zone is visually distinct and separate from the unreacted reagents allowing for the use of reaction indicators exhibiting only minor changes as well as extremely high concentrations of reactants. In addition, staged, multiple reagents can be incorporated. Whole blood can be used as a sample without the need for separate cell separating materials. Finally, the invention is useful for the detection of analytes in a broad variety of materials such as milk, environmental samples, and other samples containing target analytes.

U.S. Pat. No. 7,267,799 to Borich, et al. discloses an optical reading system, a universal testing cartridge, and a method of coupling optical reading systems. In a particular illustrative embodiment, the optical reading system includes a universal test cartridge receptor, test format determination logic, test criteria determination logic, and an optical reader module. The universal test cartridge receptor is responsive to a universal test cartridge having a test strip inserted therein. The test format determination logic determines an optical test format of the test strip. The test criteria determination logic determines an optical test criteria based upon the optical test format. The optical reader module is configured to capture an optical test image of the test strip.

U.S. Pat. No. 7,425,302 to Piasio, et al. discloses a lateral flow chromatographic assay format for the performance of rapid enzyme-driven assays. A combination of components necessary to elicit a specific enzyme reaction, which are either absent from the intended sample or insufficiently present therein to permit completion of the desired reaction, are predeposited as substrate in dry form together with ingredients necessary to produce a desired color upon occurrence of the desired reaction. The strip is equipped with a sample pad placed ahead of the substrate deposit in the flowstream, to which liquid sample is applied. The sample flows from the sample pad into the substrate zone where it immediately reconstitutes the dried ingredients while also intimately mixing with them and reacting with them at the fluid front. The fluid front moves rapidly into the final “read zone” wherein the color developed is read against predetermined color standards for the desired reaction. Pretreatment pads for the sample, as needed, (e.g. a lysing pad for lysing red blood cells in whole blood) are placed in front of the sample pad in the flow path as appropriate. The assay in the format of the invention is faster and easier to perform than analogous wet chemistry assays. Specific assays for glucose-6-phosphate dehydrogenase (“G-6PD”), total serum cholesterol, .beta.-lactamase activity and peroxidase activity are disclosed.

U.S. Pat. No. 7,521,260 to Petruno, et al. discloses an assay test strip includes a flow path, a sample receiving zone, a label, a detection zone that includes a region of interest, and at least one position marker. The at least one position marker is aligned with respect to the region of interest such that location of the at least one position marker indicates a position of the region of interest. A diagnostic test system includes a reader that obtains light intensity measurement from exposed regions of the test strip, and a data analyzer that performs at least one of (a) identifying ones of the light intensity measurements obtained from the test region based on at least one measurement obtained from the at least one reference feature, and (b) generating a control signal modifying at least one operational parameter of the reader based on at least one measurement obtained from the at least one reference feature.

U.S. Patent Application Publication No. 2009/0155921 to Lu, et al. discloses a method and apparatus for reading test strips such as lateral flow test strips as used for the testing of various chemicals in humans and animals. A compact and portable device is provided that may be battery powered when used remotely from the laboratory and, may store test data until it can be downloaded to another database. Motive power during scanning of the test strip is by means of a spring and damper that is wound by the operator during the insertion of a test strip cassette holder prior to test.

U.S. Patent Application Publication No. 2010/0311181 to Abraham, et al. discloses an assay reader system incorporating a conventional assay reader, for example a lateral flow reader, and an insert aligned with the reader's sensor to detect an assay result. The insert may include a housing that defines a cavity to receive a removable barrier, wherein the removable barrier can be aligned between the sensor and the test strip. The barrier may include an optical window, and may be cleanable and/or disposable to maintain the accuracy of the reader. Test strips are introduced into the reader through a receiving port within the insert's housing. An air inlet on the insert further maintains the reader's accuracy by allowing air to be tunneled over the housing to remove excess dust, debris, or the like.

The current methods described above for the assessment of oxidative stress, antioxidant capacity and inflammation have multiple drawbacks, including: some of the biomarkers (such as most oxidized lipids) are not stable for prolonged periods, even when stored frozen; some biomarkers (e.g. isoprostanes, widely regarded as biomarkers for oxidative stress) are generated ex vivo from the precursor (arachidonic acid) when some biological samples (particularly blood) are exposed to oxygen in the air; most require blood, which is invasive and requires a skilled person to collect the sample; most of the exotic testing laboratories have very high fees so that a multi-analyte assessment of healthy may cost from $2,000 to over $10,000, and typically requires a physician to analyze and interpret the data. Furthermore, some available tests, such as a commercial test marketed for monitoring lipid hydroperoxides in urine (it should be noted: free radicals themselves are so short-lived that they can't be directly measured in biofluids), do not employ any method to adjust or normalize the analysis for the relative concentration of the urine sample.

Furthermore, the levels of many of the biomarkers employed to assess oxidative stress, inflammation and/or antioxidant activity are impacted by and respond rapidly to factors unrelated to an individual's overall health and risk for contracting diseases. For example, the level of reactive oxygen species and consequently the levels of many biomarkers for oxidative stress, including isoprostanes and malondialdehyde, increase rapidly albeit transiently as a consequence of physical exercise. The level of nitric oxide metabolites (nitrate and nitrite) are transiently elevated following the consumption of processed foods containing nitrates as preservatives. The levels of urinary proteins can also be elevated by physical exercise. The level of isoprostanes in the urine is further influenced by the rapid metabolism of isoprostanes by the body, with the mechanism(s) and extent of metabolism of isoprostanes subject to considerable variation among individuals. Since uric acid is one of the major antioxidants present in blood and urine, the antioxidant activity of a sample is subject to variations in the rate of purine catabolism and also to dietary factors. For example, it has been reported that the primary mechanism responsible for the increase in antioxidant activity following consumption of apples is the uric acid derived from the apples. Hence, although there is significant evidence that the levels of specific individual biomarkers for oxidative stress, inflammation and/or antioxidant activity are related to health and disease risk based on extensive studies in experimental animals and in human populations, confounding factors such as those listed above are among the reasons why the application of these biomarkers for the assessment of the health and disease risk of individual humans has been very restricted.

Therefore, there is a need for a set of tests to quantify these biomarkers for these important physiological conditions, preferably including multiple biomarkers to significantly reduce confounding effects associated with the use of a single biomarker, that signal an individual's health and relative resistance to multiple diseases that can preferably be performed non-invasively for low cost and can provide accurate results regarding the health of the user. There is especially a need for a set of tests to monitor cattle health, as well as other ruminant's health.

### SUMMARY OF THE INVENTION

The present invention provides for a panel for monitoring levels of biomarkers in ruminants, including at least one inflammation monitoring test, at least one oxidative stress monitoring test, at least one antioxidant activity monitoring test, and a normalization mechanism for urine concentration.

The present invention also provides for a method of monitoring the health of ruminants, by collecting a urine sample from the ruminant, applying the sample to an assay panel, performing inflammation monitoring test(s), oxidative stress monitoring test(s), and antioxidant activity monitoring test(s) in the panel, performing normalization on urine concentration, and thereby determining the levels of biomarkers related to inflammation, oxidative stress, and antioxidant activity and therefore determining the ruminant's relative health and susceptibility to certain diseases.

### DESCRIPTION OF THE DRAWINGS

Other advantages of the present invention are readily appreciated as the same becomes better understood by reference to the following detailed description when considered in connection with the accompanying drawings wherein:

FIG. 1 is an example of a computer-generated report of the panel of the present invention;

FIG. 2 is an example of a computer-generated report of the panel of the present invention for a healthy individual;

FIG. 3 is an example of a computer-generated report of the panel of the present invention for an individual who smokes and has high OS and INF levels;

FIG. 4 is a diagram of an overview of how chronic inflammation and oxidative stress are interrelated; and

FIG. 5 is a chart of ruminant taxonomy.

### DETAILED DESCRIPTION OF THE INVENTION

The present invention provides a panel for monitoring, preferably non-invasively, the levels of biomarkers in individuals, such as humans or animals, and preferably in ruminants. Most generally, the panel includes of a set of chemical, immunochemical and/or enzymatic assays or tests that can be used together for monitoring the levels of a set of biomarkers for three conditions: inflammation, oxidative stress, and anti-oxidant activity. More preferably, the panel includes five biomarkers (two oxidative stress biomarkers, two antioxidant capacity biomarkers, a generalized inflammation biomarker), as well as a single normalizing agent to account for urine concentration on a single urine strip.

The term “assay” as used herein refers to a procedure that determines the amount of a particular constituent of a mixture or sample. “Assay” can interchangeably be used with the term “test” herein.

The term “biomarker” as used herein refers to a substance, such as, but not limited to, a protein, DNA sequence, RNA sequence, or other biological substance or substances (antioxidant activity tests can measure one specific substance or several—e.g. CUPRAC) that, when detected, indicates a particular healthy or unhealthy state of an individual.

The term “healthy” as used herein refers to a state of a person or animal that is free from detectable disease and is in good health and has a relatively low risk of developing certain diseases. Such a person or animal is considered “well”.

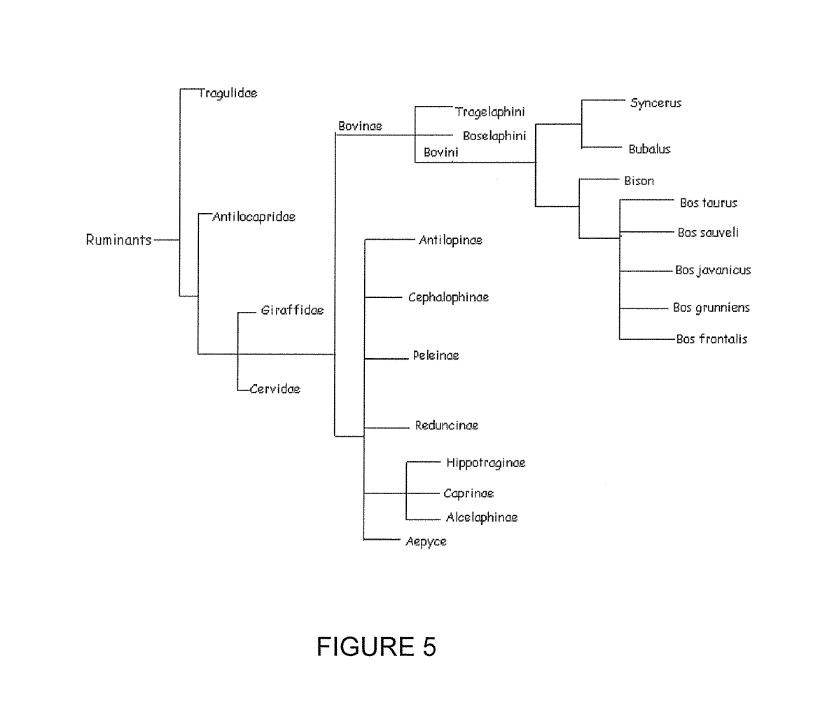

The term “ruminant” as used herein refers to a hoofed mammal from the suborder Ruminantia. Ruminants are cloven-hoofed, cud-chewing quadrupeds, such as, but not limited to, cattle, goats. sheep, yaks, bison, buffalo, deer, antelopes, giraffes, camels, llamas, okapis, pronghorn, and chevrotains. Most preferably, the ruminants in the present invention are dairy cattle and beef cattle. Ruminants eat quickly and store masses of grass (grazers) or foliage (browsers) in the rumen, i.e. first stomach, and soften the grass or foliage. The ruminants later regurgitate the softened cud and chew it again to break down cellulose, and the cud subsequently goes to the other stomach chambers to be further digested. The biomarkers in the present invention are commonly found in the urine of all ruminant species. Though the relative concentrations of each biomarker can be distinct within a species—or even subspecies—they are, nevertheless, still present and remain a valuable tool for the analysis of metabolic efficiency and for establishing phenotypic classifications of the animals based on a pre-disposition for the development of future disease states. The taxonomical breakdown of ruminants is found in FIG. 5.

The term “sample” as used herein refers to a biological sample from a human or animal and is preferably urine. Other samples can be used in the present invention in the same manner described herein, such as, but not limited to, blood, plasma, tears, and cerebral spinal fluid (CSF). While urine is specifically referred to in the description herein, it should be understood that the other types of samples can be interchanged where appropriate and the invention is performed in the same manner. It should be noted that certain biomarkers can be present in one type of sample but not in others and that the biomarker measured can be specific to a urine sample, a blood sample, etc.

The panel of the present invention represents a significant departure from traditional clinical diagnosis, which seeks to diagnose diseases. The focus of the panel is to assess, preferably by a non-invasive quantitative test, how healthy or well an individual is by monitoring biomarkers for three factors, two of which are directly related to risk of disease (oxidative damage and inflammation) and one (antioxidant activity) which is inversely related to the risks of chronic diseases such as cancer, CVD, neurodegeneration, among others. A panel comprised of tests for one or more biomarkers for all three of these factors has not been previously used, especially in a urine test, nor has a panel comprised of tests for biomarkers for these conditions been combined previously with body mass index calculations and/or an individual's lifestyle.

The initial test panel is drawn from several hundred tests that have been reported in the literature for the measurement of oxidative damage, antioxidant power and inflammation (see Table 1 for summary of published biomarkers). Selection criteria include the reliability, selectivity, and sensitivity of each component test, the stability of the analyte(s) (e.g. relatively low reactivity with air and/or light once the specimen is collected, relatively low reactivity with other components of the sample such as reactivity with proteins to form adducts or the proteolytic degradation of protein analytes), and the ease of quantifying the analytes without the need for sophisticated equipment (e.g. LC/MS). The tests in the panel can be any single test below or combinations thereof.

TABLE 1
Possible Wellness Biomarkers and Assays

| | Used as a biomarker in: | |
|---|---|---|
| Oxidative Damage: | Blood | Urine |
| Broad measures of damage | | |
| TBARS | x | x |
| Organic Hydroperoxides | x | x |
| Protein Carbonyls | x | x |
| Measure of damage to specific molecules | | |
| Lipids | | |
| Malonaldehyde | x | x |
| 4-hydroxynonenal | x | x |
| Lipid hydroperoxides | x | x |
| Isoprostanes | x | x |
| Linoleic acid oxidation products | x | x |
| Proteins | | |
| Protein carbonyls | x | x |
| Nitrotyrosine | x | x |
| Nitrothiols | x | x |
| Up to 100 other oxidized AA | x | x |
| Nucleic acids | | |
| 8-hydroxy-deoxyguanosine | x | x |
| M1dG | x | x |
| Oxidized derivatives of ribose ring | x | x |
| Small molecules and ions | | |
| Selenium | x | x |
| GSH or GSSG and the GSH/GSSG ratio | x | x |

Antioxidant Power: Used as a biomarker in blood or urine:
Direct methods (measure reaction with a redox probe)
CUPRAC (cupric reducing antioxidant capacity)
Total Antioxidant Capacity (copper-bathocuprione method)
Indirect methods (measure resistance to oxidation of a probe by an
added oxidizer)
FRAP (ferric reducing ability of plasma)
TRAP (total reactive antioxidant potential)
ORAC (oxygen radical absorbance capacity)
HORAC (hydroxyl radical antioxidant capacity)
Measurement of molecules that contribute to the total antioxidant capacity
GSH or GSSG and the GSH/GSSG ratio
Glutathione Peroxidase
Superoxide Dismutase
Uric acid
Ascorbic acid

| | Used as a biomarker in | |
|---|---|---|
| Inflammation: | blood | urine: |
| Cytokines | | |
| TNF-α | x | — |
| IL-6 | x | x |
| IL-8 | x | x |
| Several others | x | — |
| Other proteins | | |
| Osteopontin | x | x |
| Orosomucoid | — | x |
| Albumin | — | x |
| α1-microglobulin | — | x |
| Eicosanoids | | |
| PGE2 and metabolites | x | x |
| PGF2α and metabolites | x | x |
| Other molecules | | |
| Nitric oxide byproducts (NOx)(nitrate + nitrite) | x | x |
| Urinary proteins | no- | x |
| Histamine | x | x |

In a preferred embodiment, all of the biomarkers for an initial wellness screen are substances that can be quantified quickly by chemical or enzymatic reactions that do not require the use of antibodies, so that they can be incorporated into test panels that can be performed on simple chemical analyzers and/or incorporated into dry chemistry dipsticks that can be exposed to the specimen and subsequently quantified using a reflectance instrument similar to those that are widely available for other analytes. Alternatively, in other embodiments one or more of the biomarkers selected for inclusion in the panel can require the use of antibodies, including lateral flow immunoassays or immunoassays requiring the use of colorimetric, radiometric, fluorometric or chemiluminescent methods, or use more complicated analysis method(s) when collecting and/or quantifying samples in the liquid phase, such as microfluidic technologies, or microplate methods with automated or manual analysis in high throughput diagnostic machines. It should be understood that while it is preferable for one method in a single device to be employed to detect and analyze the biomarkers in all three tests, each test can also use a different method. For example, one biomarker can be analyzed by immunoassay in a microplate, and another can be analyzed by a chemical indicator. When on a single device, preferably the tests are physically separate, such as having test pads on a hydrophobic backing dipstick material and blotting excess fluid for minimal crosstalk. However, having the tests on a single device can save time in obtaining results.

Whereas the analysis of oxidative stress, antioxidant and inflammatory biomarkers has previously been performed primarily using blood specimens, the preferred embodiment of the present invention employs urine specimens that can be obtained non-invasively by a less skilled individual and with less risk of exposure to blood-borne pathogens. Further, the levels of some of the biomarkers can be substantially altered for blood samples by release of constituents of red blood cells in hemolyzed specimens, or by the ex vivo oxidation of precursors (e.g. unsaturated lipids) upon exposure of blood to air. The panel of the present invention significantly reduces the generation of ex vivo artifacts and minimizes risks of alteration.

The panel of tests, preferably performed on urine specimens, provides a more robust assessment of an individual's health status than any of the individual components. More specifically, the panel includes at least tests for at least one biomarker each for inflammation, oxidative stress, and anti-oxidant activity, that are performed in the liquid phase (in test tubes or microplate wells), adapted to a simple dipstick method employing dried reagents as described above, or incorporated into a microfluidic or a lateral flow immunoassay device.

Oxidative stress is examined via the relative concentration of reactive oxygen species. The association between abnormal levels of reactive oxygen species (i.e. oxidative stress) and various disease states is well documented. For example, Celi describes biomarkers of oxidative stress found in ruminants including MDA, TBARS, F2-Isoprostane, ORAC, FRAP, TEAC, TRAP, ROMs, and BAP (Immunopharmacology and Immunotoxicology, 2010, 1-8). Kataria, et al. (Journal of Stress Physiology & Biochemistry, Vol. 8, No. 4, 2012, pp. 72-77) describes biomarkers for oxidative stress in sheep such as vitamin A, vitamin C, vitamin E, glutathione, catalase, superoxide dismutase, glutathione reductase, and xanthine oxidase.

The oxidative stress test can include incorporating either a specific malondialdehyde (MDA) or 4-hydroxyonenal (4HNE) method to quantify lipid peroxidation and/or a thiobarbituric acid reactive substances (TBARS) method to measure a broader range of substances oxidized to aldehydes and ketones due to the actions of free radicals. MDA at varying concentrations has been shown to have a high correlation to various disease states and is an excellent biomarker due to this predictive ability. These tests are known in the art with ruminants and can be performed by an appropriate analyzing mechanism. For example, MDA and TBARS were measured and indicated that moderate hot summer weather had an effect on the oxidative status on diary cows (Bernabucci, et al., J. Dairy Sci. 85:2173-2179).

The MDA method can employ a Knoevenagel-Type Condensation reaction that is monitored at 670 nm (where few other substances absorb light) and the absence of a need to heat the sample, makes this test potentially more reliable than the TBARS assay and provides a very important confirmation of results obtained using TBARS methods. The reaction reaches an end point within 1 minute at the nominal operating temperature of the instrument, after which the color developed can be measured by reflectance at 670 nm. The value obtained is normalized to the concentration of creatinine in the sample. The test can detect MDA in urine down to approximately 3 micromolar and exhibits a strong linear response up to at least 100 micromolar. Healthy individuals have levels ranging from 15-178 nM/mM of creatinine.

TBARS can be used wherein the incubation of a urine specimen with acid and TBA at 60 degrees C. gives rise to colored products. The products are quantified by monitoring the reflectance at 530 nm kinetically over the initial 3 minutes of the reaction and determining the slope by least squares regression analysis. Since heating of urine with acid, even in the absence of TBA can give rise to products that reflect light at this wavelength, the slope of the increase in reflectance at 530 nm obtained for a blank sample (urine+acid but without TBA) is subtracted from that obtained in the presence of TBA. The net slope due to specific reactivity with TBA is then normalized to the concentration of creatinine in the urine sample. The test can detect TBARS reactivity in urine down to approximately 1 micromolar and exhibits a strong response up to at least 25 micromolar. When values are normalized to the concentration of creatinine, healthy individuals have been reported to have TBARS levels ranging 0.28-0.5 μM TBARS/mM creatinine (0.208+/−0.128 mM). Since the TBARS method involves heating urine with acid, and is read at a wavelength at which urine and products derived from heating it are colored, it can be critical to subtract a blank value without TBA to ensure that the value obtained is not an artifact due to other substances in urine. The test can be modified to reduce the strength of the acid and the temperature, thereby further reducing the color due to other urinary components reacting with acid. Bile acids, carbohydrates, nucleic acids, certain antibiotics, and amino acids that react with TBA can be reduced as artifacts by this kinetic method of analysis.

Several other biomarkers can be used to test for oxidative stress and non-limiting examples are listed in Table 1 above. High levels of these biomarkers indicate that oxidative stress is occurring in an individual. Low levels of these biomarkers indicate a healthy individual. Examples of ranges are given in the FIGURES for both oxidative damage and oxidative stress calculated from oxidative damage and total antioxidant power.

The ability for an individual to manage excess oxidative stress directly correlates with its ability to continue to function with metabolic efficiency. Antioxidant status is linked to oxidative stress, though the relationship is often inverted. When a test system defines these two categories, a much more complete and accurate picture of an individual's metabolic state emerges. Furthermore, the measurement of antioxidant status along with oxidative stress can help to eliminate the improper categorization of an individual into a metabolic phenotype, compared to the categorization based on oxidative stress levels alone. Nayyar, et al. describe that evaluation of total antioxidant capacity is necessary in conducting physiological, biochemical, and nutritional studies in ruminants (Iranian Journal of Veterinary Research, Shiraz University, Vol. 11, No. 1, Ser. No. 30, 2010).

The total antioxidant capacity assay quantifies the combined action of all antioxidants present in the sample reduction from Cu2+ following formation of a stable Cu+-cuproine complex that can be quantified at 480 nm. The redox potential for this reaction is ideal for the accurate determination of the combined antioxidant activity in a specimen that results from all of its constituents including vitamins, proteins, glutathione, uric acid, etc. The reaction reaches an end point within 2 minutes at the nominal operating temperature of the instrument, after which the color developed is measured by reflectance at 465 nm. The value obtained is normalized to the concentration of creatinine in the sample. The dipstick test can detect antioxidant activity in urine down to 0.1 mM and exhibits a strong response up to 2 mM when expressed in uric acid equivalents. Healthy individuals have TAC levels averaging 0.484+/−0.163 mM trolox equiv/mM Cr whereas TAC values are significantly lower (P<0.001) in obese individuals (P<0.001).

Oxidative stress occurs when an abnormal level of reactive oxygen species (ROS), such as lipid peroxide, lead to damage of molecules in the body. ROS can be produced from fungal or viral infection, ageing, UV radiation, pollution, excessive alcohol consumption, and cigarette smoking among other diseases. ROS can further cause age-related macular degeneration and cataracts. The antioxidant power test, sometimes called the antioxidant capacity test, employs the CUPRAC (cupric reducing antioxidant capacity) method for measuring the sum of the antioxidant activity due to multiple species (uric acid, proteins, vitamins, dietary supplements) that are present in a urine sample (Özyürek, M., Güçlü, K. and Apak, R., The main and modified CUPRAC methods of antioxidant measurement. Trends in Analytical Chemistry, 30: 652-664 (2011)). Alternatively, or additionally, modified methods can be used to specifically measure or to discriminate among uric acid, ascorbic proteins or other substances that contribute to the overall antioxidant power, thereby monitoring what is referred to as the “antioxidant reserve.” These tests are known in the art and can be performed by an appropriate analyzing mechanism. Several other biomarkers can be used to test for antioxidant power and non-limiting examples are listed in Table 1 above. Most of these tests require incubating the sample with a probe that changes on oxidation and then adding a radical generator. The longer it takes for the probe to change, the more antioxidant capacity there is. The CUPRAC method, and other methods that employ a redox indicator that directly measures the reaction of antioxidants with substances with appropriate redox potential to effect a color change. A higher value for antioxidant power, i.e. a greater amount of the biomarkers for antioxidant power, indicates a healthy individual because the individual has compounds that can neutralize free radicals that cause oxidative damage and stress. Examples of ranges of antioxidant power are shown in the FIGURES.

As in humans, systemic, low-grade inflammation in ruminants is a contributing cause of several disease states. Early recognition and intervention to reduce/eliminate this inflammation not only improves the current metabolic efficiency of the animal, but it also serves to protect the animal and delay/halt the onset/progression of new disease states. For example, inflammation is indicated in mastitis in dairy cattle (Celi, R. Bras. Zootec., v. 39, p. 348-363, 2010).

Inflammation is comprised of a complex series of physiological and pathological events, including the increased production of several proteins (e.g. cytokines such as IL-6 and IL-8, as well as COX-2 and the inducible form of nitric oxide synthase). The production of nitric oxide (NO), by the inducible isoform of nitric oxide synthase can increase up to 1000 times during inflammation, and has been shown to be a useful biomarker for inflammation (Stichtenoth, D., Fauler, J., Zeidler, H., Frolich, J. C. Urinary nitrate excretion is increased in patients with rheumatoid arthritis and reduced by prednisolone Annals of the Rheumatic Diseases 54:820-824 (1995)). Because NO is relatively unstable, the production of NO can be tested by employing methods for the measurement of it degradation products nitrate and nitrite, i.e. measuring nitrite or the sum of nitrite and nitrate in a blood or urine sample, which are often abbreviated as NOx. These tests are known in the art and can be performed by an appropriate analyzing mechanism. Further, although very high levels of protein in urine are associated with kidney disease, it is known that the retention of blood proteins by the kidney is reduced by the effect of certain inflammatory cytokines, so that modest elevations in the levels of urinary proteins that are less than those associated with kidney disease can be used as a biomarker for inflammation. Several other biomarkers can be used to test for inflammation and non-limiting examples are listed in Table 1 above. Higher levels of inflammation biomarkers indicate that inflammation is occurring in an individual, possibly indicative of disease. Lower levels of inflammation biomarkers indicate a healthy individual. Examples of ranges of inflammation biomarkers are shown in the FIGURES. Chronic inflammation can lead to hay fever, atherosclerosis, and rheumatoid arthritis. Anti-inflammatory agents have also been shown to significantly reduce the incidence of heart disease, diabetes, Alzheimer's disease, and cancer.

A NO test can be based on reduction of nitrate to nitrite and the quantification of the total (nitrate+nitrite) in the sample, an approach that is widely used for the reliable quantification of NOS activity biological fluids. The reaction can reach an end point within 2 minutes at the nominal operating temperature of the instrument, after which the color developed is measured by reflectance at 575 nm. The value obtained is normalized to the concentration of creatinine in the sample. The test can detect the total nitrate and nitrite levels in urine down to approximately 10 micromolar and exhibits a strong response up to at least over 100 micromolar. Healthy individuals typically have levels ranging from 25-125 μM/mM of creatinine.

A ketone test can be included to indicate metabolic efficiency. When ruminants are not able to consume the necessary nutrients to maintain a basic level of metabolic health, additional energy is obtained through the breakdown of internal fats and proteins (muscle). The breakdown of muscle by an animal leads to the production of ketone bodies, and the detection and concentration of these ketone bodies can be used to indicate the overall health and metabolic status of the animal. Furthermore, with frequent testing and early detection of these metabolites, it is possible to intervene in a timely manner, before lasting damage has occurred to the animal in question.

An acetone test can be included to indicate metabolic efficiency. Acetone can be produced during starvation and, when found in ruminants, it is indicative of a negative energy balance. When specifically examining cows, the presence of acetone in urine can be used to indicate a negative energy balance in the third week post-partum. During this period, cows are unable to absorb sufficient nutrients to meet their metabolic needs for milk production and self-maintenance (Garrido-Delgado, et al. Talanta, 78 (2009) 863-868).

A urinary protein test can be used that allows for the detection of even modest elevations in urinary protein levels. The assay reaction reaches an end point within less than 5 seconds at the nominal operating temperature of the instrument, after which the color developed is measured by reflectance at 550 nm. The value obtained is normalized to the concentration of creatinine in the sample. The dipstick test can detect protein in urine down to approximately 30 microgram/mL and exhibits a strong response up to at least 250 micrograms/mL. Healthy individuals have levels ranging from 0.03-0.26 micrograms/mg creatinine. The presence of proteins within the urine can be used as a broad indication of overall health, from categories ranging from hydration status to kidney health.

The combination of the oxidative stress test, antioxidant power test, and inflammation test in this particular panel is unique. Pairs of these tests have been combined in the prior art. For example, Basu (Basu, S. Bioactive Eicosanoids: Role of Prostaglandin F2 and F2-Isoprostanes in Inflammation and Oxidative Stress Related Pathology. Mol. Cells 30: 383-391 (2010)) and others have monitored urinary biomarkers for oxidative stress and inflammation. Others have monitored antioxidant power and oxidative stress and computed an index for an individual's oxidative status (Vassalle C, Pratali L, Boni C, Mercuri A, Ndreu R. An oxidative stress score as a combined measure of the pro-oxidant and anti-oxidant counterparts in patients with coronary artery disease. Clin Biochem. 41:1162-7 (2008)). The use of biomarkers for oxidative stress (e.g. Isoprostanes like Basu uses) has been reported to be an independent risk factor for CVD. The use of antioxidant power and oxidative damage markers has been reported on frequently. Cutler, et al. (Ann. N.Y. Acad. Sci. 1055:136-158(2005)) lists a large number of biomarkers for all three parameters and proposes that a large number of assays for this large number of biomarkers, employing both serum and urine (some technically very demanding, some not very reliable) to assess an individual but does not further provide guidance in the practical application and interpretation of this list of tests. However, while all three parameters of oxidative stress, antioxidant power, and inflammation have been mentioned together in the prior art, it has been within the context of a large listing of assays and not exclusively with regards to a practical method suitable for wide-spread application, in particular a non-invasive panel that can be performed using a set of tests on a urine specimen. Importantly, these research applications have not found their way into simple and widely useful testing methods.

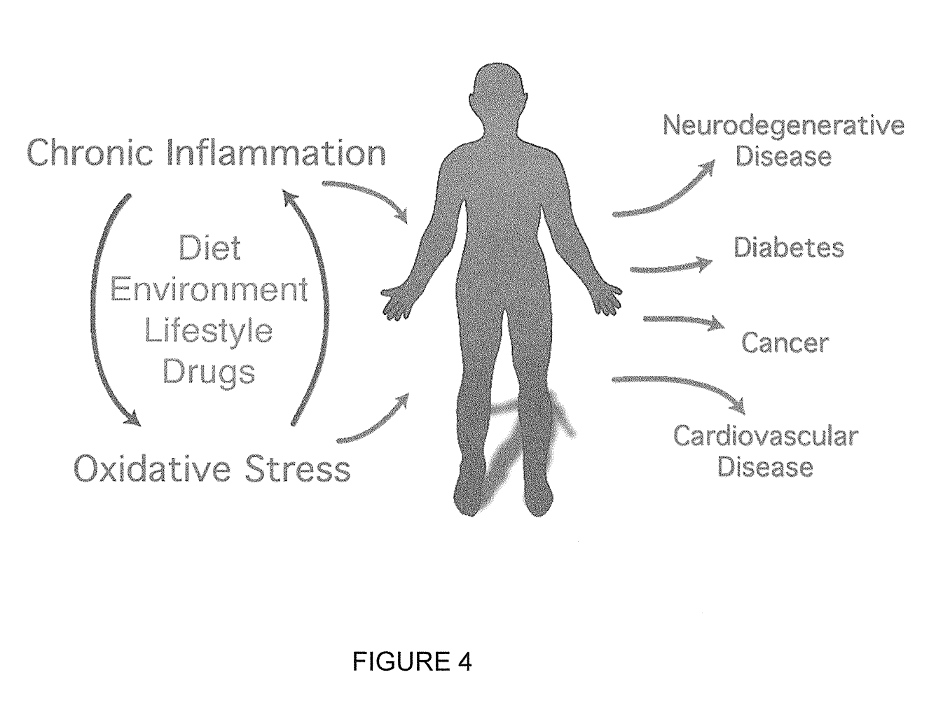

In the ten years since the sequencing of the human genome, it has become increasingly apparent that, while genetics plays a major role in the development of diseases for a small percentage of the population, the overall impact of genetics on major non-infectious diseases in humans is only about 15-20%. Much more important, especially for the development of the diseases that account for most morbidity and mortality in developed countries (chronic diseases such as cancer, cardiovascular diseases, neurodegenerative and autoimmune diseases) are the impact of diet, lifestyle (including exercise, smoking, alcohol use) and the environment. All of these factors influence an individual's health and, as illustrated in FIG. 4, they result in increases or decreases in inflammation and/or oxidative stress. Moreover, the oxidative stress can trigger some reactions that increase the level of inflammation.

The importance of oxidative stress to human health is evidenced by thousands of scientific publications and hundreds of biomarkers that have been reported for oxidative damage, as well as the development of several tests for antioxidant activity and the widespread application of one (the ORAC test) to measure the antioxidant activity in foods and juices, and the enormous market for nutraceutical supplements that have antioxidant activity in vitro. However, as has been now clearly demonstrated in the case of vitamin E, antioxidant activity in vitro does not necessarily translate into a change in the level of oxidative stress in vivo.

In keeping with traditional medical practices, some biomarkers for inflammation and oxidative damage have been translated individually into clinical practice. C-reactive protein is increasingly recognized inflammatory biomarker in blood (but not urine) that is used to monitor for development of cardiovascular disease. Levels of one specific protein, measured as the albumin/creatinine ratio, in urine is used clinically to measure microalbuminuria, with the increased levels of this specific protein associated with elevated risk for kidney and cardiovascular diseases. Similarly, elevated isoprostane levels (oxidative damage biomarkers in blood or urine) have been reported to be independent risk markers for cardiovascular disease with statistics comparable to CRP or HDL/LDL ratio, but isoprostane measurements are typically complex and have not found wide-spread application. However, the use of antioxidant power has been only applied to human biofluids in academic research studies, and the use of panels incorporating multiple biomarkers have been restricted to inflammatory biomarkers or oxidative stress biomarkers, typically without inclusion of antioxidant markers, and typically including inflammatory and oxidative stress markers only in very large, expensive, broad panels that include 20 or more biomarkers with comprehensive analysis or interpretation of the results referred to a physician.

The incorporation of a small number of relatively broad tests for oxidative damage and inflammation with a broad test for antioxidant activity provides, for the first time, a relatively rapid, broad, and affordable screening panel to assess an individual's wellness and susceptibility to major chronic diseases. By including information regarding their body mass index, and/or information regarding the test subject's age, lifestyle and disease history, and linking the numerical results to a database of specific interpretive narratives drawn from the scientific literature regarding the import of the data and methods (including specific diets, exercise, etc) to improve the values relative to a person's age, the panel provides an unprecedented approach to improved screening of broad populations for health and wellness, and for the feedback needed to help effect behavioral changes to improve health.

The panel can also include a normalization agent or mechanism for urine concentration. The concentration of substances in urine can vary widely, depending on an individual's consumption of water, sweat, etc. Methods that allow for adjustment for urinary output include (a) performing studies on first morning specimens (most concentrated, but inconvenient, still variable and not always reliable), (b) collection of a 24-hour urine specimen (very reliable but very inconvenient and rarely used anymore), and (c) normalization of values to a metabolite that is excreted at a relatively constant rate or to the specific gravity of the specimen. Among the latter, creatinine is most commonly used. There are relatively few conditions for which the use of creatinine for normalization of the levels of substances in urine is not 100% accurate. Therefore, normalization of values to the concentration of creatinine is very common in clinical medicine, in medical research and there are several established methods for performing the assay. Therefore, all of the values related to oxidative stress, antioxidant power, and inflammation are divided by the creatinine concentration. This simple process significantly improves the reliability and reproducibility and permits the tracking of changes in an individual's wellness over time and as the result of changes in diet, lifestyle, etc.

For example, the total daily output of creatinine is approximately 1.2 g for a human. The average daily urine volume is 1.2 L (range: 600-1600 mL), so the mean creatinine concentration is approximately 1 mg/mL. Based on this average, creatinine correction can adjust the urine concentration of a given analyte to an average concentration of 1 mg/mL. During the course of a day, some samples can have a concentration above 1 mg/mL and others can be below 1 mg/mL, but the analyte concentration can be corrected to a value theoretically equivalent to the value of a urine specimen that has a concentration of 1 mg/mL.

Alternatively, if specific gravity is used, when paired with pH can also be used to determine hydration status and the risk of developing kidney stones. pH can also be used to identify possible shortcomings within the diet that can ultimately lead to the development of various disease states.

Since it is also known that biological specimens, in particular urine, absorb light and that the color of a specimen is dependent on many endogenous substances as well as substances ingested in the diet or as medications, the panel can further include an adjustment mechanism for adjusting of the measurement for specific biomarker tests to eliminate to correct for color or fluorescence due irrelevant substances in the sample. For example, one position on the test strip can be read immediately and used as a blank for subtraction of any background color in urine at 465 nm (for the TAC assay), and also kinetically monitored at 550 nm as the sample is heated with acid to correct for interfering substances in the TBARS assay.

The panel can further include a data entry mechanism for entering an individuals age, height, and weight to calculate an individual's body mass index (BMI), as well as information regarding the individual's lifestyle (e.g. tobacco and/or alcohol use) or condition and health of the animal and other factors. Since it is well documented that antioxidant activity declines with age and that oxidative stress tends to increase with age, age-related normalization can also be performed on the results. The BMI can be used in comparisons with the results of the three tests of the panel, i.e. BMI versus oxidative damage, BMI versus antioxidant power, BMI versus oxidative stress (OS) status, BMI versus inflammation, further described below. The BMI can be compared to the test results in order to determine risk for diseases.

The panel can also include a quantification device for analyzing test results as well as an output mechanism for displaying the results. These components and their use are further described below.

The panel of the present invention is used in the following method. The panel is used by collecting a sample from a ruminant (preferably urine), applying the sample to the panel, performing the tests for at least one biomarker for each of the three conditions described above, normalizing the values to correct for the relative concentration of the specimen and determining the levels of these biomarkers for health related to inflammation, oxidative stress, and antioxidant activity.

A sample for analysis by the panel is easily obtained from an individual ruminant's urine or other body fluid described above. The sample can be obtained by a cup to collect liquid for the microfluidic format or, most preferably, by a dipstick that is placed in the urine for the dipstick format. The urine can then be applied to the panel by inserting the dipstick therein. A strip can also be placed in the individual's urine stream while urinating.

The urine sample can optionally be treated with a substance that helps to preserve the components being measured from decomposition during storage or shipment, and/or prevents the generation of additional reactive substances outside of the body, and/or retards the growth of microbes in the specimen that might alter the values during storage or shipment. These additive(s) do not themselves alter the values of the tests involved in the panel. However, preferably, the sample is analyzed as soon as possible after collection to reduce the decomposition or further reactions of biomarkers in the panel.

Analysis of one or more biomarkers, preferably two each for oxidative stress and inflammation to improve reliability and reduce errors associated with confounding factors that can influence specific biomarkers, for each of the three conditions is performed as specified above by the panel. When a dipstick is used, detecting a color change in the dipstick can indicate the measurement of specific analytes or biomarkers in each test of the panel. Each test can change the amount of colored light reflected from one of the components of the dipstick. For a negative result (i.e. the presence of a biomarker is not detected), the strip can remain its original color, or it can change to a specific color. For a positive result (i.e. the presence of a biomarker is detected), the strip can change to a distinctively different color than the negative result. One example is the strip turning blue for a negative result and pink for a positive result. In preferred embodiments, the results are non-qualitative (color versus lack of color) but vary in degree corresponding to the level of the biomarker present. For example, an intense color can indicate the presence of high levels of the specified biomarker, and a muted color can indicate the presence of low levels of the biomarker.

Subsequently, the dipstick or other dry chemistry device can be inserted into an instrument that quantifies the reflected color for each test pad (preferably handheld) and a quantitative value can be recorded. In this method, the amount of each biomarker present can be determined to provide further information as to the health of the user. In other words, lower or higher levels of biomarkers, and not just their presence, can be relevant to the state of health. Alternatively, a quantification device is included in the panel itself and is not a separate device.

The quantification device can include or be coupled to a computer with software that is capable of performing analysis using the data thus obtained with an analyzing mechanism. The analyzing mechanism can compute values of each of the biomarkers in the tests, perform normalization as described above, as well as compute relationships of the test results with each other, the test results with BMI described above or, after calculating oxidative stress and antioxidant power, the ratio of both can be calculated to determine OS (oxidative stress) status and this value can be compared with BMI or inflammation. The analyzing mechanism can also search a database for facts relating high or low levels of specific biomarkers to disease risks, and can include facts derived from scientific literature that provide suggestions for lifestyle changes, or suggestions for further testing based on the test results, and combinations thereof. The analyzing mechanism can indicate risk for ruminant diseases, such as those for cattle, i.e. mastitis, transition cow syndrome, or bovine respiratory disease complex.

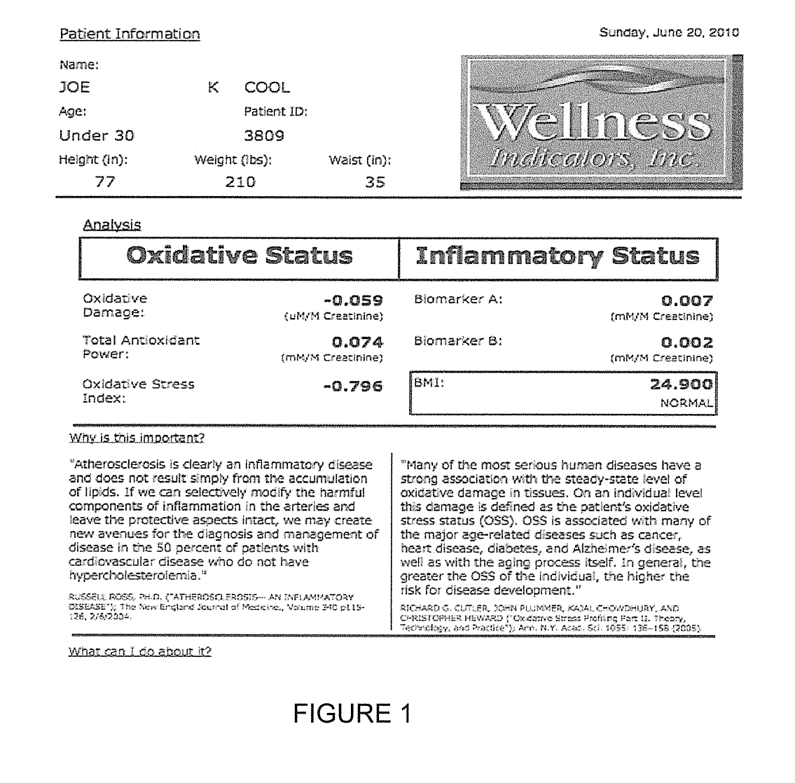

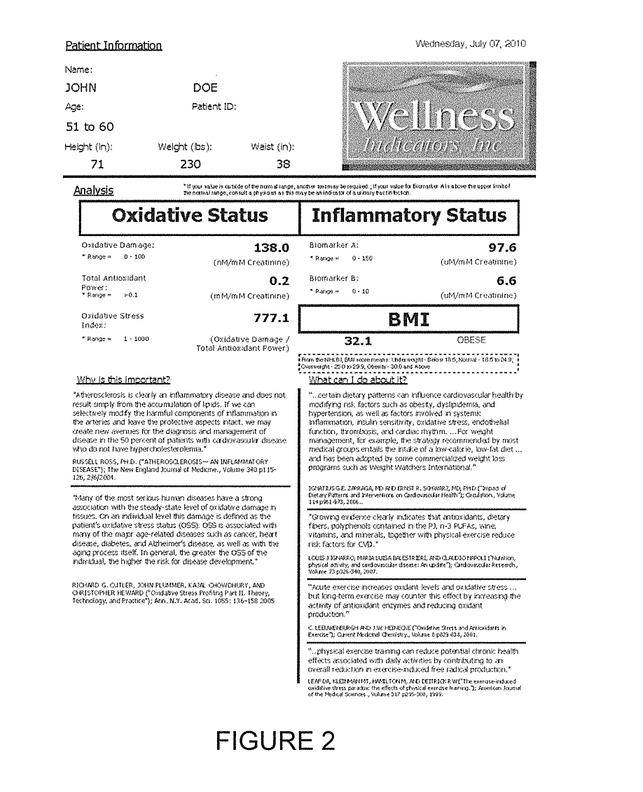

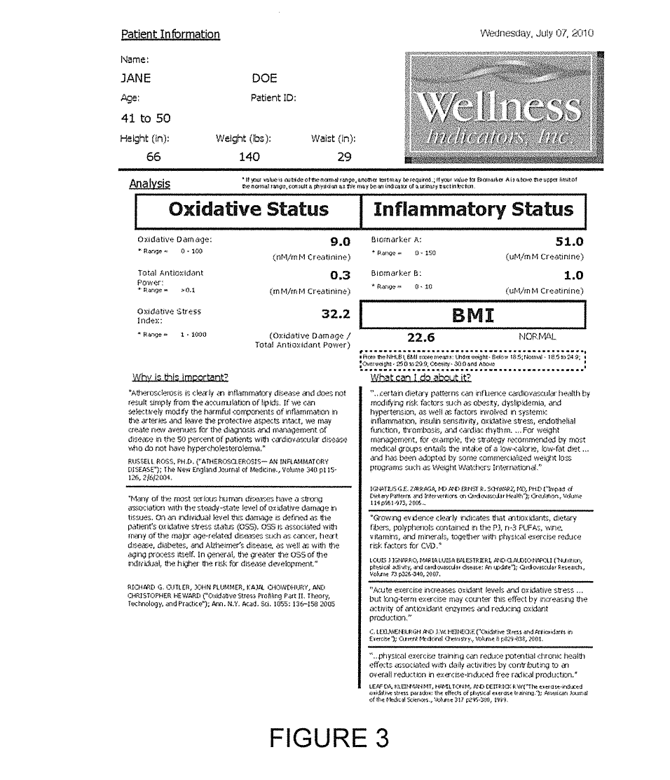

The presence of biomarkers for health can then be indicated to the user. The quantification device further includes an output mechanism to display the results in a meaningful way to an individual, veterinarian, or health care practitioner. The display can be on a screen included on the panel and can include a printing mechanism for printing the results. Alternatively, the output mechanism can also send the results over wireless signals or wires to a PDA, smart phone, or a remote computer for print out or display. The results can be incorporated into a report on an individual's wellness that includes, but is not limited to, the results of the tests, comparison to the values and ratios computed to normal ranges that have previously been established for normal healthy men and women of different ages, ethnicities (if relevant) and/or other relevant parameters. Such a report can also incorporate historical data for an individual subject that was obtained using the same method(s). The report can further show the information from the database described above. Examples of such a report are shown in FIGS. 1-3.

Most preferably, for use with ruminants, the urine is analyzed by inserting the dipstick or strip into a handheld reader device that provides a numerical readout of the strip's test sites. The reader device includes light emitting diodes (LEDs), photo sensors, and a PLC that compiles the wavelength reflections into a numeric value displayed on a LCD screen on the reader device. The numeric display shows the values in numerals, but the results can also be color-coded as red (disease state), yellow (potential problem), and green (healthy), so that untrained personnel can recognize a problem with the ruminant or other individual.

The panel of the present invention is useful for testing as part of wellness programs administered by insurance companies or large insurers, by employers, by clinicians, nutritionists, wellness consultants, and others as well as fitness and training programs administered by sports organizations or the military. The preferred use of the panel is a point of testing health and wellness assessment, which can be performed in a doctor's office, by a health care practitioner or an insurance agent after suitable training. The panel can also be used by individuals to monitor their health in their own home.

The panel of the present invention including the three tests on a single, easy-to-use, and disposable test strip provides better results than individual assays for the various biomarkers discussed herein. Tests for inflammation, oxidative stress, antioxidant activity have been studied independently and in controlled studies for large numbers of subjects, each has been associated with disease and/or disease risk. Oxidative stress and inflammation often increase or decrease together, and it is known that certain transcription factors are involved in this. e.g. oxidative stress turns on the expression of some genes encoding some inflammatory proteins and vice versa. However, each of the specific tests for oxidative stress and inflammation biomarkers is subject to some confounding factors as discussed above. Hence, elevated urinary protein can result from strenuous exercise or athletic training and not inflammation (although overexertion can cause inflammation); NOx may be falsely and transiently elevated by eating some hot dogs; MDA will transiently increase following athletic training—but endogenous sources for antioxidant activity are increased by exercise. By comparison to one's lipid profile, it is much more informative to measure a panel of biomarkers, just as one's cholesterol or HDL level alone does not provide as complete and accurate a picture. There are multiple endogenous and exogenous variable that can confound any of the assays in TABLE 1. By employing a panel with more than one but a manageable number of markers, one can improve the reliability of the overall panel versus one test or even one test for each condition.

Furthermore, with the integration of all of these tests onto a single platform, it is now possible to aggregate the data from all results and to compile it in a complementary way so that the data from individual tests enhances the interpretation of other tests on the strip. For example, the fact that a ruminant animal demonstrates a high level of oxidative stress may or may not be indicative of a wider metabolic issue when viewed alone, but when combined with information detailing a low total antioxidant status and high level of systemic inflammation, it is now possible to identify the animal as being in a poor metabolic state and furthermore, depending on the ruminant in question, possibly correlate those readings with a disease state. For example, using dairy cows entering the lactation period, it is possible to identify an animal as highly susceptible to Transition Cow Syndrome prior to irreversible damage occurring and to treat the animal, allowing it to become a fully functioning member of the herd again.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A panel for monitoring levels of biomarkers in ruminants, comprising at least one inflammation monitoring test, at least one oxidative stress monitoring test, at least one antioxidant activity monitoring test, and a normalization mechanism for urine concentration.

2. The panel of claim 1, wherein said inflammation monitoring test quantifies biomarkers chosen from the group consisting of TNF-α, IL-6, IL-8, osteopontin, orosomucoid, albumin, α1-microglobulin, PGE₂, PGF_{2α}, nitric oxide, nitrate and nitrate derived from nitric oxide (NOx), histamine, ketones, acetones, urinary protein and combinations thereof.

3. The panel of claim 1, wherein said oxidative stress monitoring test quantifies biomarkers chosen from the group consisting of protein carbonyls, thiobarbituric acid reactive substances (TBARS), malonaldehyde, 4-hydroxynonenal, lipid hydroperoxides, isoprostanes, linoleic acid oxidation products, nitrotyrosine, nitrothiols, 8-hydroxy-deoxyguanosine, M1dG, oxidized derivatives of the ribose ring, selenium, GSH, GSSG, the GSH/GSSG ratio, and combinations thereof.

4. The panel of claim 1, wherein said antioxidant activity monitoring test is chosen from the group consisting of CUPRAC (cupric reducing antioxidant capacity), a test based on a copper cuprione redox indicator, FRAP (ferric reducing ability of plasma), TRAP (total reactive antioxidant potential), ORAC (oxygen radical absorbance capacity), HORAC (hydroxyl radical antioxidant capacity), and combinations thereof.

5. The panel of claim 1, wherein said antioxidant activity monitoring test quantifies a biomarker chosen from the group consisting of uric acid, GSH, GSSG, GSH/GSSG ratio, glutathione peroxidase, superoxide dismutase, ascorbic acid, and combinations thereof.

6. The panel of claim 1, wherein at least two biomarkers are measured in said inflammation monitoring test and at least two biomarkers are measured in said oxidative stress monitoring test.

7. The panel of claim 1, wherein said panel of tests is performed on urine.

8. The panel of claim 1, wherein said panel includes a dry chemistry dipstick that incorporates at least one of said inflammation monitoring test, said oxidative stress monitoring test, and said antioxidant activity monitoring test.

9. The panel of claim 1, wherein said panel includes a lateral flow immunoassay incorporating at least one of said inflammation monitoring test, said oxidative stress monitoring test, and said antioxidant activity monitoring test.

10. The panel of claim 1, wherein said panel includes a dry chemistry dipstick and a lateral flow immunoassay incorporating at least two of said inflammation monitoring test, said oxidative stress monitoring test, and said antioxidant activity monitoring test.

11. The panel of claim 1, wherein said panel includes at least one liquid phase analytical test chosen from the group consisting of immunoassays, lateral flow immunoassays, colorimetric immunoassays, radiometric immunoassays, fluorometric immunoassays, chemiluminescent immunoassays, test tubes, microplate wells, and combinations thereof.

12. The panel of claim 1, further including a mechanism to adjust for the inherent color or fluorescence of the biofluid being analyzed.

13. The panel of claim 1, further including a data entry mechanism for entering information about the ruminant.

14. The panel of claim 1, further including at least one device for the quantification of the levels of the biomarkers and an output mechanism for displaying test results, exporting test results to a computer for further computations, and producing printed reports.

15. A method of monitoring the health of ruminants and relative risk for developing disease(s), including the steps of:
collecting a urine sample from the ruminant;
applying the urine sample to an assay panel;
performing at least one inflammation monitoring test, at least one oxidative stress monitoring test, and at least one antioxidant activity monitoring test in the panel;
performing normalization on urine concentration; and
determining levels of biomarkers related to inflammation, oxidative stress, and antioxidant activity and therefore determining the ruminant's health.

16. The method of claim 15, wherein the ruminant is chosen from the group consisting of cattle, goats. sheep, yaks, bison, buffalo, deer, antelopes, giraffes, camels, llamas, okapis, pronghorn, and chevrotains.

17. The method of claim 15, wherein said applying step is further defined as applying the sample to a mechanism chosen from the group consisting of a lateral flow microfluidic device, test tubes, microplate wells, a lateral flow immunoassay device, and a dry chemistry dipstick.

18. The method of claim 15, wherein said collecting step further includes a step chosen from the group consisting of preserving the sample from decomposition, preventing generation of additional reactive substances, retarding growth of microbes in the sample, and combinations thereof.

19. The method of claim 15, wherein the inflammation monitoring test quantifies biomarkers chosen from the group consisting of TNF-α, IL-6, IL-8, osteopontin, orosomucoid, albumin, α1-microglobulin, PGE2, PGF2α, nitric oxide, nitrate and nitrate derived from nitric oxide (NOx), histamine, ketones, acetones, urinary protein, and combinations thereof.

20. The method of claim 15, wherein the oxidative stress monitoring test quantifies biomarkers chosen from the group consisting of protein carbonyls, thiobarbituric acid reactive substances (TBARS), malonaldehyde, 4-hydroxynonenal, lipid hydroperoxides, isoprostanes, linoleic acid oxidation products, nitrotyrosine, nitrothiols, 8-hydroxy-deoxyguanosine, M1dG, oxidized derivatives of the ribose ring, selenium, GSH, GSSG, the GSH/GSSG ratio, and combinations thereof.

21. The method of claim 15, wherein the antioxidant activity monitoring test is chosen from the group consisting of CUPRAC (cupric reducing antioxidant capacity), a test based on a copper cuprione redox indicator, FRAP (ferric reducing ability of plasma), TRAP (total reactive antioxidant potential), ORAC (oxygen radical absorbance capacity), HORAC (hydroxyl radical antioxidant capacity), and combinations thereof.

22. The method of claim 15, wherein the antioxidant activity monitoring test quantifies a biomarker chosen from the group consisting of uric acid, GSH, GSSG, the GSH/GSSG ratio, glutathione peroxidase, superoxide dismutase, ascorbic acid, and combinations thereof.

23. The method of claim 15, wherein said performing step is further defined as quantifying at least two biomarkers for inflammation and oxidative stress.

24. The method of claim 15, wherein said determining step includes the step of detecting a color change in a dipstick containing the sample corresponding to levels of biomarkers related to inflammation, oxidative stress, and antioxidant activity.

25. The method of claim 24, wherein said determining step further includes the step of quantifying reflected color for each test and recording quantitative value of the biomarkers.

26. The method of claim 15, wherein said determining step determines a healthy individual when low levels of inflammation biomarkers, low levels of oxidative stress biomarkers, and high levels of antioxidant activity biomarkers are detected, and said determining step determines an unhealthy individual when high levels of inflammation biomarkers, high levels of oxidative stress biomarkers, and low levels of antioxidant activity biomarkers are detected, and determines that the ruminant is at risk for developing disease.

27. The method of claim 15, wherein said normalization step further including the step of analyzing the levels of the biomarkers by computing values of the biomarkers, performing normalization, adjusting for the inherent color of the sample, and computing relationships between multiple tests.

28. The method of claim 27, wherein said performing normalization step is further defined as dividing values of the biomarkers by a value chosen from the group consisting of creatinine concentration and the specific gravity of the specimen.

29. The method of claim 15, further including the steps of calculating oxidative stress, antioxidant power, and oxidative stress, and comparing oxidative stress with BMI or inflammation.

30. The method of claim 15, further including the step of employing a database to provide information chosen from the group consisting of facts relating high or low levels of biomarkers to disease risks, suggestions for lifestyle changes, suggestions for further testing, and combinations thereof.

31. The method of claim 15, further including the step of displaying test results to a user by a mechanism chosen from the group consisting of a display on the panel, wirelessly to a PDA, wirelessly to a smart phone, and wirelessly to a remote computer.
